(19) United States
(12) Patent Application Publication
(10) Pub. No.: US 2014/0162290 A1
(43) Pub. Date: Jun. 12, 2014

(54) PROBE FOR DETECTING DEAD CELL

(71) Applicant: RIKEN, Saitama (JP)

(72) Inventors: Yasuyoshi WATANABE, Wako-shi (JP); Yasuko MATSUMOTO, Wako-shi (JP); Chinuyo SUMITA, Wako-shi (JP)

(73) Assignee: RIKEN, Saitama (JP)

(21) Appl. No.: 14/087,740

(22) Filed: Nov. 22, 2013

(30) Foreign Application Priority Data
Nov. 22, 2012 (JP) 2012-256679
May 24, 2013 (JP) 2013-109917

(51) Int. Cl. G01N 33/569; C07K 14/705

(52) U.S. Cl. CPC G01N 33/56966; C07K 14/70503
USPC 435/7.21; 530/387.3

(57) ABSTRACT

Provided is a molecular imaging probe that accumulates specifically and highly sensitively at a tumor site in vivo, and enables quantitative analysis, e.g. a probe for detecting an apoptotic cell(s) and/or a necrotic cell(s), comprising a fusion protein of a Tim4 protein and a protein or polypeptide that forms a dimer, the protein or polypeptide being bound to the C-terminus of the Tim4 protein, wherein the mucin domain of the Tim4 protein and the C-terminal side domain thereof are replaced with a polypeptide consisting of the amino acid sequence of a) or b) below: a) an amino acid sequence having a length of 30 to 120 amino acid residues comprised in the amino acid sequence of the mucin domain of a wild-type Tim4 protein; b) an amino acid sequence having an identity of not less than 80% to the amino acid sequence of a).

### BACKGROUND OF THE INVENTION

1. Technical Field

The present invention relates to a probe that detects a dead cell(s) such as an apoptotic cell(s) and/or a necrotic cell(s) and thereby enables molecular imaging of the cell(s).

2. Background Art

Cell death imaging in vivo is important for such as early assessment of efficacy therapy, prognosis of survival, early diagnosis heat failure and so on. Various imaging probes detecting cell death have been developed and used for positron emission tomography (PET) and single photon emission computed tomography (SPECT) imaging as tracers.

Examples of such molecular imaging probes include phosphatidylserine (hereinafter also referred to as PS) binding proteins. These proteins bind to PS exposed on the outer leaflet of the plasma membrane of apoptotic or necrotic cells (Apoptosis, (2010) 15, 1072-1082). More specifically, use of a radionuclide-labeled molecule of annexin A5 or C2A domain of synaptotagmin I, which are PS-binding molecules, as a tracer for SPECT or PET has been proposed.

Tim4 (T-cell immunoglobulin protein and mucin domain 4) is known as one of a protein related to immune functions and cell viability (JP 4572276 B). Tim4 is membrane-spanning protein composed of signal sequence, IgV domain with PS binding ability, mucin-like domain, transmembrane region, and cytoplasmic region (Nature (2007) 450, 435-439). Tim4 is a single transmembrane protein and is dimerized to bind to PS. Although IgV domain of Tim4 have a metal ion pocket (Immunity, (2007) 27(6), 941-951; and Immunological Reviews, (2010) 235, 172-189), Tim4 bind to PS without Ca²⁺, unlike Annexin A5 which requires Ca²⁺ for binding to PS(Nature (2007) 450, 435-439; and Immunity (2007) 27, 927-940). There has been no case where Tim4 protein was used as a molecular imaging probe.

### SUMMARY OF THE INVENTION

As imaging probe for targeting PS, Annexin A5 and C2A domain of synaptotagmin I have been used to date. They accumulate to liver and/or kidney non-specifically and conformational changes easily occur during labeling process. Further, since these molecules require high concentration (2.5 mM or more) of Ca²⁺ for binding to PS, these molecules are not suitable for performing quantitative image analysis.

In view of this, the present invention aims to provide a molecular imaging probe that specifically and highly sensitively accumulates at a tumor induced apoptosis, and enables quantitative analysis.

As a result of intensive study to solve the above problem, the present inventors developed a new imaging probe based on Tim4. The imaging probe is fusion protein of a Tim4 which have mucin-like domain of varied lengths and a protein or polypeptide for dimerization. The fusion proteins bind to apoptotic cells and/or necrotic cells specifically, and radiolabeled probe accumulated to tumor inducing apoptosis. The present inventors also discovered that deletion of C-terminal side of mucin domain increase sensitivity of cell death detection as compared to the wild type, thereby completing the present invention.

That is, aspects of the present invention are exemplified as follows.

[1] A protein as a probe for detecting an apoptotic cell(s) and/or a necrotic cell(s) (hereinafter also referred to as the “protein of the present invention”),

which is a fusion protein of a Tim4 protein and a protein or polypeptide to ensure dimerization, wherein the protein or polypeptide is fused to the C-terminus of the Tim4 protein,

wherein a part or the whole of the region consisting of the mucin domain, transmembrane domain, and cytoplasmic region in the Tim4 protein is replaced with a polypeptide consisting of the amino acid sequence of a) or b) below:

a) an amino acid sequence having a length of 30 to 120 amino acid residues consisting of a part of the amino acid sequence of the mucin domain of a wild-type Tim4 protein;

b) an amino acid sequence having an identity of not less than 80% to the amino acid sequence of a).

[2] The protein according to [1], wherein the part or the whole of the region is replaced with a polypeptide consisting of the amino acid sequence of a′) or b′) below:

a′) an amino acid sequence from the amino acid residue at the N-terminus to the amino acid residue at any one of positions 30 to 120 of the amino acid sequence of the mucin domain of a wild-type Tim4 protein;

b′) an amino acid sequence having an identity of not less than 80% to the amino acid sequence of a′).

[3] The protein according to [1] or [2], wherein the IgV domain of the Tim4 protein has the amino acid sequence of c) or d) below:

c) the amino acid sequence of the IgV domain of a wild-type Tim4 protein;

d) an amino acid sequence which is identical to the amino acid sequence of c) except that one or several amino acids are substituted, deleted, inserted, and/or added, and which has PS-binding capacity.

[4] The protein according to any one of [1] to [3], wherein the protein or polypeptide that forms a dimer is a human IgG Fc region protein.
[5] The protein according to any one of [1] to [4], wherein the wild-type Tim4 protein is a human Tim4 protein.
[6] The protein according to any one of [1] to [5], wherein the molecular weight of the fusion protein as measured by SDS-PAGE under non-reducing conditions is 100 kDa to 250 kDa.
[7] A probe, comprising the protein according to any one of [1] to [6], and being labeled (hereinafter also referred to as the “probe of the present invention”).
[8] A diagnostic imaging agent for a tumor, comprising the probe according to [7].
[9] A diagnostic imaging kit, comprising the diagnostic imaging agent for a tumor according to [8].
[10] A method for detecting an apoptotic cell(s) and/or a necrotic cell(s), comprising:

detecting an apoptotic cell(s) and/or a necrotic cell(s) by using the probe according to [7].

According to the present invention, provided is a molecular imaging probe that enables highly sensitive detection of a dead cell(s), accumulates in tumor inducing apoptosis in vivo, and enables quantitative analysis. According to the probe of the present invention, accurate detection and imaging of cell death can be performed in vivo, and the obtained result can be used for diagnosing a disease or evaluating therapeutic effect of a drug.

### BRIEF DESCRIPTION OF THE DRAWINGS

FIG. 1 shows maximum intensity projections images of animals bearing A431 tumor at 48 h after [⁶⁴Cu]mTim4-Fc or [⁶⁴Cu]mTim4-Δ230-Fc injection.

FIG. 2 shows TUNEL assay or activated-Caspase-3 immunostaining images of sections of dissected tumors (photographs). A to C, tumors were dissected from MMC-administered mice individuals; D to F, tumors were dissected from physiological saline-administered mice individuals; A and D, anti-activated caspase 3 antibody immunostaining images (red); B and E, TUNEL staining images (green); C and F, fusion images of the anti-activated caspase 3 antibody immunostaining image, the TUNEL staining image and a nuclear staining image using Hoechst 33258 (blue).

FIG. 3 shows dot blot images showing the binding ability of each hTim-Fc to PS. A filter spotted with PS, incubated with each hTim4-Fc and immunostained by anti-IgG-Fc antibody (photographs).

FIG. 4 shows dot blot images showing the binding ability of hTim4-187-Fc to various phospholipids (photographs).

FIG. 5 shows immunostaining images of apoptotic cells with hTim4-187-Fc or Annexin A5 (photographs). A, Double staining images of hTim4-187-Fc stained with anti-IgG-Fc antibody conjugated with FITC and PI; B, Double staining figures of Annexin A5-FITC and PI. Each panel shows: left column, hTim4-187-Fc or Annexin A5 staining images; middle column, PI staining images; right column, fusion images of the fluorescence images and a transmission image. The upper and lower columns show different views under the same conditions.

### DETAILED DESCRIPTION OF THE PREFERRED EMBODIMENTS

The protein for a probe of the present invention is a fusion protein of a Tim4 protein and a protein or polypeptide that forms a dimer, which protein or polypeptide is bound to the C-terminus of the Tim4 protein.

A wild-type Tim4 protein is constituted by a signal sequence, an IgV domain, a mucin domain, a transmembrane domain, and a cytoplasmic region, in the order from the N-terminal side. The IgV domain has a PS-binding site, and the mucin domain has sugar chain-binding sites.

The amino acid sequences of wild-type Tim4 proteins of human and mouse are shown in SEQ ID NO:2 and SEQ ID NO:4, respectively. These proteins have 48% amino acid sequence identity to each other.

In the protein of the present invention, a region comprising the mucin domain of Tim4 protein is replaced with a downsized mucin domain with deleted C-terminus and/or the N-terminus amino acid residues. The region to be replaced, i.e. the region comprising the mucin domain of the Tim4 protein, is a region that corresponds to a part or the whole of the region consisting of the mucin domain, transmembrane domain, and cytoplasmic region in the Tim4 protein, and at least comprises the full-length mucin domain. In view of the simplicity in construction of the fusion protein, the whole of the region consisting of the mucin domain, transmembrane domain, and cytoplasmic region can preferably be replaced.

More specifically, the downsized mucin domain can be a polypeptide consisting of: a) an amino acid sequence of 30 to 120 amino acid residues, preferably 45 to 80 amino acid residues, more preferably 50 to 60 amino acid residues, consisting of a part of the amino acid sequence of the mucin domain of a wild-type Tim4 protein; or b) an amino acid sequence having an identity of not less than 80%, preferably not less than 90%, more preferably not less than 95%, to the amino acid sequence of a). Alternatively, the polypeptide may be a polypeptide consisting of: c) an amino acid sequence having the amino acid sequence of a) except that one or several amino acids are deleted, substituted, inserted, and/or added. In the present description, the meaning of the term “several” may vary depending on the positions of the amino acid residues in the tertiary structure of the protein and the types of the amino acid residues, and is within the range in which the effect of the present invention is not largely deteriorated. More specifically, the term can mean 2 to 50, preferably 2 to 30, more preferably 2 to 10, especially preferably 2 to 5.

In view of the ease of preparation, the size of the wild type mucin domain is reduced preferably by deleting the C-terminal side thereof. That is, the region comprising mucin domain of Tim4 protein can preferably be replaced with a polypeptide consisting of: a′) an amino acid sequence from the amino acid residue at the N-terminus to the amino acid residue at any one of positions 30 to 120, preferably at any one of positions 45 to 80, more preferably at any one of positions 50 to 60, of the amino acid sequence of the mucin domain of a wild-type Tim4 protein. Alternatively, the region comprising mucin domain of Tim4 protein can preferably be replaced with a polypeptide consisting of: b′) an amino acid sequence with an identity of not less than 80%, preferably not less than 90%, more preferably not less than 95%, to the amino acid sequence of a′); or c′) an amino acid sequence which is identical to a′) except that one or several amino acids are deleted, substituted, inserted, and/or added.

Also, the size of the wild type mucin domain can be reduced by deleting the N-terminal side, or by deleting both the N-terminal side and the C-terminal side.

Downsizing of Tim4 fusion protein contributes to specific accumulation of the probes to tumor inducing apoptosis which containing target dead cells, while non-specific accumulation of the probe at other sites can be suppressed.

The amino acid sequence of the mucin domain of human Tim4 protein is shown in SEQ ID NO:6, and the amino acid sequence of the mucin domain of mouse Tim4 protein is shown in SEQ ID NO:8. When the probe of the present invention is clinically applied to diagnostic imaging or the like, the Tim4 protein employed is preferably one derived from human.

The IgV domain of the Tim4 protein in the protein of the present invention may be wild type or may be modified type. The modified type IgV domain refers to an IgV domain having an amino acid sequence which is identical to the amino acid sequence of a wild type IgV domain except that one or several amino acids are deleted, substituted, inserted, and/or added, and having PS-binding capacity. In view of maintaining the PS-binding capacity, the IgV domain preferably has a wild-type sequence.

While it is known that the inner region of the IgV domain is mainly involved in binding of the Tim4 protein to PS (Immunity, (2007) 27(6), 941-951; and Immunological Reviews, (2010) 235, 172-189), it is also considered that the portions close to the both ends of the IgV domain forms a β-sheet structure, so as to be involved in maintaining the spatial structure of the Tim4 protein, and thereby to indirectly contribute to the binding to PS.

The protein or polypeptide that forms a dimer in the protein of the present invention is bound to the C-terminus of the modified Tim4 protein. The protein or polypeptide that forms a dimer is introduced in order to promote dimerization of the Tim4 protein upon its binding to PS. That is, in other words, the protein or polypeptide that forms a dimer can mean a protein or polypeptide to ensure dimerization.

Examples of the protein or polypeptide that forms a dimer include the IgG Fc region and leucine zipper structure. The protein of the present invention also includes a fusion protein prepared by fusing an arbitrary protein or polypeptide with a Tim4 protein and further fusing two molecules of the resulting fusion protein to each other at the arbitrary protein or polypeptide comprised therein by S—S bond(s). Among them, the human IgG-Fc region is preferred in view of achieving an appropriate molecular weight to suppress non-specific accumulation of the probe at sites other than the target site and in view of the ease of purification of the protein in the later-described preparation of the probe.

The molecular weight of the protein of the present invention can be preferably 100 kDa to 250 kDa, more preferably 130 kDa to 200 kDa, still more preferably 150 kDa to 180 kDa, as measured by SDS-PAGE under non-reducing conditions.

Probe molecules administered in vivo generally tend to accumulate non-specifically in the liver in cases where the molecular weight is high, or in the kidney in cases where the molecular weight is low. Therefore, the fusion protein in the present invention preferably has the above-described size in view of achieving its specific accumulation in the site or tissue containing the target dead cell(s) while suppressing its non-specific accumulation at other sites.

The protein of the present invention can be prepared by an arbitrary method including well-known genetic engineering methods, and the method of preparation is not limited.

For example, a region(s) encoding the C-terminal side and/or the N-terminal side of the mucin domain in a DNA encoding a wild-type Tim4 is/are deleted such that a modified mucin domain having a desired amino acid length remains, to obtain a DNA fragment encoding the modified Tim4, followed by ligating the DNA fragment with a DNA fragment encoding a protein or polypeptide that forms a dimer by a well-known method, to prepare a DNA fragment encoding a fusion protein. The obtained DNA fragment encoding the fusion protein is introduced into an appropriate expression vector, and Escherichia coli (E. coli) is transformed with the resulting vector. Thereafter, the fusion protein is expressed and recovered by a well-known method, and then purified as appropriate by chromatography or the like.

Introduction of the mutation(s) such as deletion, substitution, insertion, and/or addition to the wild-type sequence can also be carried out by a well-known method.

The protein of the present invention can be provided as a probe by labeling, and the probe can be applied for detecting an apoptotic cell(s) and/or a necrotic cell(s). The type of labeling is not limited as long as it enables simple detection of a tumor site or tissue containing an apoptotic cell(s) and/or a necrotic cell(s). The labeling can be appropriately carried out by a well-known method, and examples of the method include fluorescence labeling using FITC or the like; enzyme labeling using peroxidase or the like; radioisotope (RI) labeling using a nuclide usually employed in diagnostic imaging such as PET and SPECT; and biotin labeling.

In view of application to a commonly used diagnostic imaging method such as PET or SPECT, the probe of the present invention is especially preferably RI-labeled. Examples of the PET nuclide include, but are not limited to, ⁶⁴Cu, ⁸⁹Zr, ⁶⁸Ga, ¹²⁴I, and ¹⁸F. Examples of the SPECT nuclide include, but are not limited to, ¹²³I, ¹¹¹In, and ^{99m}Tc.

The protein of the present invention can specifically bind to PS among the various phospholipids present in the cell membrane. The binding capacity of the protein of the present invention is equivalent to that of Annexin A5, which is a known PS-binding protein. In general, a labeled substance has a decreased binding capacity to its target molecule as compared to the binding capacity of the substance before labeling. However, the protein of the present invention maintains its binding capacity to PS even in the mode of a probe prepared by labeling with RI or the like.

According to the above-described properties, the probe of the present invention binds to PS of a cell(s) in which the PS appears on the cell membrane surface, and thereby enables detection of such a cell(s). In general, PS is present in the inner cell membrane in living cells, but PS appears on the cell membrane surface in dead cells. Therefore, the probe of the present invention can specifically detect a dead cell(s). More specifically, the probe of the present invention can detect an apoptotic cell(s) and a necrotic cell(s). The probe can also detect a cell(s) at a stage where the cell(s) is/are dying due to apoptosis induction or the like, and a cell(s) that has/have already died.

Since the protein of the present invention is a fusion protein comprising a modified Tim4 protein, administration of the protein of the present invention in vivo does not cause non-specific accumulation thereof in the kidney, while the protein of the present invention specifically accumulates at a site or tissue containing an apoptotic cell(s) and/or a necrotic cell(s).

Further, since the binding of the protein of the present invention to PS is not dependent on the Ca²⁺ concentration, quantitative analysis is possible even in cases where the probe of the present invention is administered in vivo.

Thus, in the mode as a probe, the protein of the present invention can be a molecular imaging probe that enables highly sensitive detection of a dead cell(s), accumulates specifically at the tumor site exhibiting cell death in vivo, and further enables quantitative analysis. In particular, by appropriately labeling the protein of the present invention with RI or the like, the protein can be used as a tracer in diagnostic imaging such as PET or SPECT. Therefore, the probe of the present invention can be included in a diagnostic imaging agent for a tumor exhibiting cell death, and the diagnostic imaging agent can also be provided in the mode of a diagnostic imaging kit.

Examples of the embodiment of the probe of the present invention to be used as a tracer in diagnostic imaging such as PET or SPECT include application to diagnosis of diseases such as cancer and application to evaluation of therapeutic effects of drugs. The type of the cancer or tumor tissue is not limited.

When a method for detecting an apoptotic cell(s) and/or a necrotic cell(s), or a tumor tissue comprising such a cell(s) is carried out using the probe of the present invention, the method can be carried out according to a general method.

In cases of in vitro detection, for example, the probe of the present invention can be added to sample cells to react them for an appropriate period of time, and then the detection can be carried out according to a detection method selected depending on the type of the label.

In cases of in vivo detection, the probe of the present invention can be administered to a target individual by a means such as injection (e.g. local or systemic injection) or infusion into a vein, and, after an appropriate period of time, for example, 1 to 24 hours after the administration, radiation can be measured by SPECT or PET to perform diagnosis or evaluation. The methods and techniques for the diagnosis and the tests can basically be identical to those for normal diagnosis and tests for cancer, or normal diagnostic imaging.

The dose of the probe of the present invention for administration to an individual is not particularly limited, but can be preferably 10 to 100 μg/individual, more preferably 15 to 74 μg/individual in cases of human. In cases of an RI-labeled probe, the probe can be administered such that the dose is 50 to 250 MBq/individual, preferably 40 to 200 MBq/individual, more preferably 37 to 185 MBq/individual.

The dose for mouse can be preferably 500 to 700 μg/kg. In cases of an RI-labeled probe, the dose for mouse can be preferably 10 to 25 MBq/individual.

### EXAMPLES

### Example 1

### Preparation of Wild-Type or Modified mTim4-Fc

By the following procedure, a fusion protein of a wild-type or modified mouse Tim4 and a human IgG Fc region protein was prepared.

E. coli (JM109, Takara Bio Inc.) was transformed with a plasmid vector encoding the sequence information of a fusion protein of a wild-type mouse Tim4 and a human IgG Fc region protein (mTim4-Fc), pTim4-Fc (provided by Prof. Shigekazu Nagata; see Nature (2007) 450, 435-439 for the preparation method), so as to amplify the plasmid vector. The plasmid vector was purified using FastPlasmid Mini Kit-250 preps (V Prime), and PCR was carried out using the obtained pTim4-Fc as a template and Expand High Fidelity PCR System (Roche). Using the primers of SEQ ID NOs:11 and 12, a DNA fragment encoding mTim4-ΔM230-Fc (a modified type fusion protein comprising a mTim4 part in which the C-terminal side of the mucin domain of the wild-type mTim4 is deleted so that the amino acid sequence from the N-terminus to the 96th amino acid of the mucin domain remains) was prepared. Further, using the primers of SEQ ID NOs:11 and 13, a DNA fragment encoding mTim4-ΔM184-Fc (a modified type fusion protein comprising a mTim4 part in which the C-terminal side of the mucin domain of the wild-type mTim4 is deleted so that the amino acid sequence from the N-terminus to the 50th amino acid of the mucin domain remains) was prepared. These PCR products and pTim4-Fc were each digested with restriction enzymes SalI and EcoRV (both were manufactured by Takara Bio Inc.), and subjected to agarose gel electrophoresis. The bands of interest were each cut out from the gel and purified. The purified PCR product was ligated to the purified pTim4-Fc, and E. coli was transformed with the ligation product. The transformed E. coli was inoculated on an LB plate supplemented with ampicillin (0.1 mg/mL ampicillin; 1.5% agarose; 40 capsules/L of Circle grow, Q-BIO gene), and incubated at 37° C. for 16 hours. Using a formed colony as a template, the primers of SEQ ID NOs:14 and 15, and EmeraldAmp MAX PCR Master Mix (Takara Bio Inc.), colony PCR was carried out to check the transformant, and then DNA sequence analysis was carried out. Using each plasmid vector of interest comprising DNA encoding mTim4-Fc, mTim4-ΔM230-Fc, or mTim4-ΔM184-Fc, expression of each fusion protein was carried out according to the following method. The DNA sequence, the amino acid sequence, and the number of amino acid residues in the mucin domain, of each prepared protein to be expressed are shown in Table 1.

TABLE 1
Sequence of each fusion protein

| | Number of | | |
|---|---|---|---|
| | amino acid | | |
| | residues | | |
| | in the | | |
| | mucin | DNA | Amino acid |
| Fusion protein | domain | sequence | sequence |
| mTim4-Fc | 138 | SEQ ID NO: 9 | SEQ ID NO: 10 |
| mTim4-ΔM230-Fc | 96 | SEQ ID NO: 16 | SEQ ID NO: 17 |
| mTim4-ΔM184-Fc | 50 | SEQ ID NO: 18 | SEQ ID NO: 19 |

*In each sequence, the human IgG Fc region is omitted.

E. coli transformed with the plasmid vector encoding the fusion protein was cultured with shaking in 200 mL of LB medium supplemented with 0.1 mg/mL ampicillin at 37° C. for 16 hours, and the amplified vector was purified using EndoFree Plasmid Maxi Kit (QIAGEN). The purified vector was transfected into 293T cells by the calcium phosphate method, and the medium was replaced with DMEM medium 24 hours later. The medium was collected 48 hours after the medium replacement, and centrifugation was carried out at 1000×g for 5 minutes, followed by collecting the supernatant. Protein A agarose beads (Thermo Scientific) were added in an amount of 25 μL per 50 mL of the collected medium, and the resulting mixture was rotated at 4° C. overnight. On the next day, the beads were recovered, and washed 5 times with PBS (150 mM NaCl, 20 mM phosphate buffer, pH7.0). The beads were then suspended in 100 μL of 100 mM glycine buffer, pH 3.0, and left at room temperature for 5 minutes statically. Thereafter, centrifugation was carried out and the supernatant was collected. Using Amicon Ultra (Millipore, 30 kDa cut), the collected supernatant was subjected to buffer exchange to PBS and concentrated, and then stored at −20° C. until use. As a result, 100 to 150 μg of each fusion protein was obtained from 200 mL of the culture supernatant.

### Reference Example 1

### PET Imaging

By the following procedure, the fusion proteins obtained as described above (mTim4-Fc, mTim4-ΔM230-Fc, and mTim4-ΔM184-Fc) were each labeled with RI.

The stored solution of each fusion protein was subjected to buffer exchange to PBS (D-PBS, Wako Pure Chemical Industries, Ltd.). An aqueous solution of 10 mM p-SCN-Bn-NOTA (NOTA, Macrocyclics) adjusted to pH7.9 to 8.4 with 0.1 N NaOH was added to each fusion protein at an amount of 1000 equivalents, and the resulting mixture was left at 4° C. overnight statically. Thereafter, the reactant was applied to a desalting column (PD-10, Thermo Sci.) equilibrated with PBS, the fusion protein bound to NOTA was separated from unbounded NOTA. The NOTA-binding fusion protein fraction was concentrated using Amicon Ultra, and was subjected to buffer exchange to 100 mM acetate buffer (pH 6.5). Thereafter, 6 MBq of an aqueous solution of [⁶⁴Cu]CuCl₂ per 1 μg of the NOTA-binding fusion protein was added thereto, and the resulting mixture was incubated at 40° C. for 30 minutes to perform the chelating reaction of ⁶⁴Cu. Subsequently, unreacted ⁶⁴Cu was removed by 3 times of washing with 300 μL of 100 mM glycine solution (pH 6.5) using Amicon Ultra, solution exchange to 0.05% Tween 20-PBS (PBS-T) was performed, and then the protein concentration was measured. As a result, an RI-labeled product with a purity of not less than 90% and a specific activity of 600 to 800 MBq/nmol was obtained for each fusion protein (Table 2).

TABLE 2
Specific activity and purity of ⁶⁴Cu-labeled mTim4 protein

| | Specific activity | |
|---|---|---|
| | (MBq/nmol) | Purity (%) |
| [⁶⁴Cu]mTim4-Fc | 597.1 ± 141.2 | 93.8 ± 1.5 |
| [⁶⁴Cu]mTim4-ΔM230-Fc | 622.6 ± 164.5 | 92.4 ± 2.3 |
| [⁶⁴Cu]mTim4-ΔM184-Fc | 782.8 ± 116.2 | 93.3 ± 1.5 |

For a PET imaging experiment, model animals were prepared as follows.

Male Balb/c-Ajcl-nu/nu mice of 5 to 6 weeks old (CLEA Japan, Inc.) were provided, and divided into two groups (the MMC administration group and the vehicle group; 3 individuals per group). A431 cells were inoculated into the left femoral region of each mouse, 2 to 3 weeks before the PET experiment for the MMC administration group, or 10 days to 2 weeks before the PET experiment for the vehicle group. Individuals of which the tumor size became 50 to 120 cm³ on the day before the administration of the RI-labeled product were subjected to the PET experiment. One day, 3 days, and 5 days before the administration of the RI-labeled product, MMC (mitomycin C) was administered via the tail vein at an amount of 5 mg/6.25 mL/kg for the MMC administration group, and physiological saline was administered at amount of 6.25 mL/kg for the vehicle group.

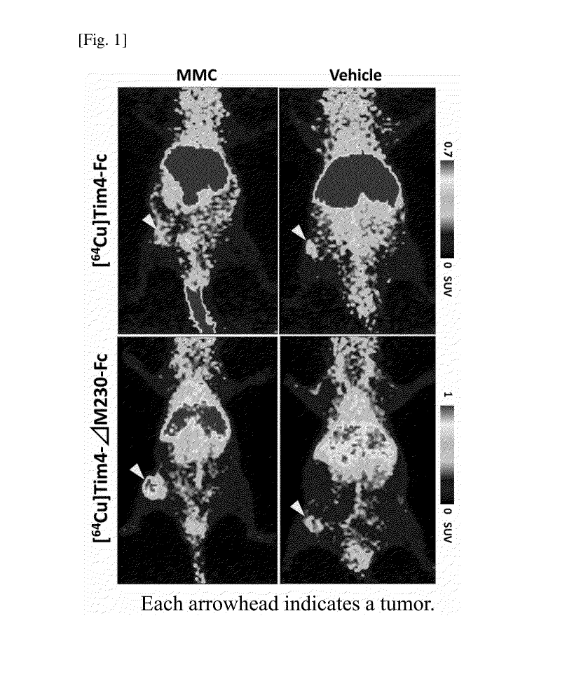

Each of the ⁶⁴Cu-labeled products prepared as described above was administered via the tail vein at 0.2 to 0.25 mg/kg (15 to 25 MBq/body), and, 48 hours after this administration, dynamic imaging was carried out for 1 hour (MicroPET Focus 220, Siemens). The obtained images are shown in FIG. 1.

After completion of imaging of all mice, the mice were dissected, and the radioactivity in each tissue was measured with a γ-counter (Table 3).

As a result, in the cases of administration of [⁶⁴Cu]mTim4-Fc, no difference was found between the mice given MMC and the mice given the physiological saline. However, in the cases of administration of [⁶⁴Cu]mTim4-ΔM230-Fc or [⁶⁴Cu]mTim4-ΔM184-Fc, accumulation of the probe in the tumor was found in the mice given MMC.

TABLE 3
Radioactivity distribution in organs and tissues

| | mTim4-Fc | | mTim4-ΔM230-Fc | | mTim4-ΔM184-Fc | |
|---|---|---|---|---|---|---|
| | MMC | Vehicle | MMC | Vehicle | MMC | Vehicle |
| Urine | 2.12 ± 0.95 | 1.08 ± 0.90 | 1.84 ± 1.38 | 2.81 ± 1.16 | 2.05 ± 1.07 | 1.33 ± 0.66 |
| Blood | 1.33 ± 0.80 | 0.73 ± 0.31 | 1.72 ± 2.19 | 2.63 ± 2.66 | 2.74 ± 1.69 | 4.02 ± 2.96 |
| Heart | 0.50 ± 0.26 | 0.41 ± 0.25 | 0.57 ± 0.46 | 0.80 ± 0.49 | 0.84 ± 0.39 | 0.75 ± 0.59 |
| Lung | 1.10 ± 0.61 | 0.85 ± 0.74 | 1.84 ± 2.60 | 1.31 ± 0.86 | 1.90 ± 1.02 | 1.15 ± 0.90 |
| Spleen | 1.40 ± 0.77 | 0.99 ± 0.28 | 2.17 ± 1.47 | 1.05 ± 0.59 | 2.60 ± 1.96 | 1.28 ± 0.63 |
| Pancreas | 0.34 ± 0.16 | 0.20 ± 0.11 | 0.36 ± 0.32 | 0.42 ± 0.24 | 0.62 ± 0.32 | 0.38 ± 0.28 |
| Kidney | 1.73 ± 0.51 | 1.42 ± 0.18 | 2.08 ± 1.17 | 2.12 ± 0.89 | 2.42 ± 0.81 | 1.94 ± 0.92 |
| Gallbladder | 1.39 ± 0.02 | 1.11 ± 0.60 | 1.21 ± 0.44 | 1.26 ± 0.36 | 2.49 ± 1.34 | 1.43 ± 0.49 |
| Liver | 11.13 ± 2.68 | 7.30 ± 1.29 | 14.00 ± 3.87 | 6.76 ± 1.31 | 14.69 ± 1.64 | 9.20 ± 0.78 |
| Intestine | 0.62 ± 0.21 | 0.44 ± 0.22 | 0.72 ± 0.48 | 0.66 ± 0.20 | 1.05 ± 0.66 | 0.72 ± 0.45 |
| Muscle | 0.18 ± 0.08 | 0.14 ± 0.07 | 0.22 ± 0.12 | 0.32 ± 0.25 | 0.32 ± 0.17 | 0.27 ± 0.23 |
| A431 | 1.24 ± 0.62 | 1.23 ± 0.50 | 2.66 ± 2.04 | 1.76 ± 1.04 | 2.90 ± 1.35 | 0.73 ± 0.06 |

(% ID/g ± Standard deviation)

### Reference Example 2

### Diagnosis of Apoptosis in Target Tissue

After the completion of the PET experiment, the tumor was isolated from each mouse, and frozen sections were prepared. The prepared frozen sections were fixed with cold methanol, and stored at −20° C. until use. The sections were used for the experiment within 3 to 5 days after the dissection.

The frozen sections were subjected to blocking using Protein Block, Serum-Free (Dako), and then subjected to TUNEL staining using DeadEnd Fluorometric TUNEL System (Promega). Thereafter, a 1000-fold diluted primary antibody (Cleaved Caspase-3(Asp175)Antibody, CST) was react with the sections at room temperature for 2 hours, and a secondary antibody (Cy3-conjugated anti-rabbit IgG antibody) was then reacted with the sections at room temperature for 2 hours. Also, nuclear staining was performed using Hoechst 33256 (Dojindo). Thereafter, the samples were observed under a confocal laser microscope.

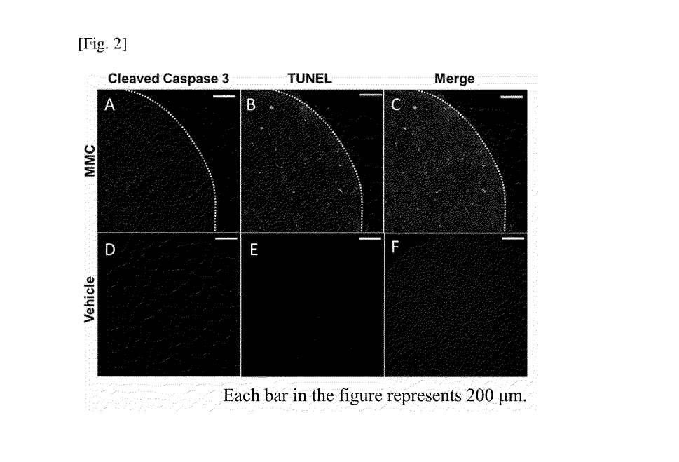

The results are shown in FIG. 2. In the frozen sections of the tumor isolated from the MMC-administered individuals, cells positive for the activated caspase 3 antibody and TUNEL were observed over the whole area of the tumor. By contrast, these markers were negative in the sections from physiological saline-administered individuals. Thus, occurrence of apoptosis in the tumor of the MMC-administered individuals was confirmed.

### Example 2

### Preparation of Wild-Type and Modified hTim4-Fc

By the following procedure, a fusion protein of human Tim4 (the wild type, and modified types with mucin domains having various lengths) and a human IgG Fc region protein was prepared.

Using a vector encoding the hTim4 sequence (pCMV6-XL5 Homo sapiens T-cell immunoglobulin and mucin domain containing 4, transcript variant 1 as transfection-ready DNA, OriGene Technologies) as a template, and a primer containing an EcoRV restriction site at 5′-end (SEQ ID NO:22) and a primer containing a BglII restriction site at 3′-end (SEQ ID NO:23), PCR was performed. The obtained PCR product and pFUSE-hIgG1e3-Fc2 (InvivoGen) encoding the human IgG-Fc region were each digested with EcoRV and BglII, and the resulting digestion products were ligated to each other. E. coli (JM109, Takara Bio Inc.) was transformed with the resulting ligation product, and colonies were obtained. From the obtained colonies, plasmids were extracted, and the DNA sequences of the plasmids were analyzed. A colony having the hTim4 sequence inserted therein was subjected to large-scale culture, to obtain a vector. The obtained vector was amplified using the primers of SEQ ID NOs:20 and 21, and the obtained PCR product was inserted into a protein expression vector (pcDNA3.3-TOPO, Life technologies). E. coli (TOP10, Life technologies) was transformed with the vector obtained by the insertion, the direction of the insertion was confirmed by DNA sequence analysis, and the obtained vector was designated phTim4-Fc.

Using the primers shown in Table 4 and PrimeSTAR Mutagenesis Basal Kit (Takara Bio Inc.), vectors encoding fusion proteins having mucin domains with various lengths, in which the C-terminus and/or the N-terminus of the wild-type mucin domain was/were deleted. More specifically, phTim4-Fc was used as a template to prepare phTim4-240-Fc and phTim4-187-Fc; the obtained phTim4-187-Fc was used as a template to prepare phTim4-184-Fc, phTim4-181-Fc, phTim4-178-Fc, phTim4-175-Fc, phTim4-162-Fc, phTim4-149-Fc, phTim4-135-Fc, phTim4-130-Fc and phTim4-Δ131-187-Fc; the obtained phTim4-240-Fc was used as a template to prepare phTim4-Δ131-240-Fc; and the obtained phTim4-Δ131-187-Fc was used as a template to prepare phTim4-Δ131-187Δ241-310-Fc. The DNA sequence, the amino acid sequence, and the number of amino acid residues in the mucin domain, of each prepared protein to be expressed are shown in Table 5.

The obtained vector of 37.5 μg was transfected into 30 mL of 1×10⁶ cells/mL Freestyle 293F cells (Life technologies) using Freestyle MAX Reagent (Life technologies), and the cells were cultured with shaking at 37° C. under 8% CO₂ for 4 days. On Day 3 of the culture, 10 mL of the medium was further added. The culture supernatant was collected after 4 days cultivation, 250 μL of Protein A agarose beads (Pierce) were added to 40 mL of the collected supernatant, and the expressed protein was recovered. The recovered expressed protein was purified using a gel filtration column (Superdex 200 10/300 GL, GE healthcare), subjected to buffer exchange to PBS, pH 6.8, and stored at 4° C. until use. The purified protein was subjected to SDS-PAGE, and the molecular weight under non-reducing conditions was calculated (Table 6).

TABLE 4
Primers for preparation of the expression vector for each fusion protein

| | Forward primer (5′-3′) | Reverse primer (5′-3′) |
|---|---|---|
| Fusion protein | SEQ ID NO: Sequence | SEQ ID NO: Sequence |
| hTim4-Fc | 22: TTGATATCCGAGACTGTTGTGACGGAGGTTTTGGGTC | 23: AAAGATCTTTGGGAGATGGGCATTTCATTCTTCATTG |
| hTim4-240-Fc | 24: TTGATATCCGAGACTGTTGTGACGGAGGTTTTGGGTC | 25: ACAGATCTAGAAGTAGACTCAGCACTACTCCAGGAAT |
| hTim4-Δ131-187-Fc | 26: GAGAGCCCCATCAACATCCCACGTG | 27: GTTGATGGGGCTCTCTGTAGATTCAG |
| hTim4-Δ131-240-Fc | 26: GAGAGCCCCATCAACATCCCACGTG | 27: GTTGATGGGGCTCTCTGTAGATTCAG |
| hTim4-Δ131-187 | 28: GAGAGCCATTGCCGTCTTCACAACA | 29: ACGGCAATGGCTCTCTGTAGATTCAG |
| Δ241-310-Fc | | |
| hTim4-187-Fc | 30: GTCTTCGGATCTGTGGAGTGCCCAC | 31: CACAGATCCGAAGACGGCAATGGTTG |
| hTim4-184-Fc | 32: AACCATTGGATCTGTGGAGTGCCCAC | 33: CACAGATCCAATGGTTGTCATCTGGAGTG |
| hTim4-181-Fc | 34: TCCAGATGGGATCTGTGGAGTGCCCAC | 35: CACAGATCCCATCTGGAGTGGTGTTCCGG |
| hTim4-178-Fc | 36: AACACCAGGATCTGTGGAGTGCCCAC | 37: CACAGATCCAGGTGTTCCGGTTGTGAGAT |
| hTim4-175-Fc | 38: CACAACCGGATCTGTGGAGTGCCCAC | 39: CACAGATCCGGTTGTGAGATCGGGTGTGG |
| hTim4-162-Fc | 40: AGCTGCAGGATCTGTGGAGTGCCCAC | 41: CACAGATCCTGCAGCTGGGGTTGTTGTC |
| hTim4-149-Fc | 42: ACAAGCGGATCTGTGGAGTGCCCAC | 43: CACAGATCCGCTTGTTGTTGTTGTT |
| hTim4-135-Fc | 44: ACGCACGGATCTGTGGAGTGCCCAC | 45: CACAGATCCGTGCGTGGTTGTTGAGG |
| hTim4-130-Fc | 46: AGAGCCGGATCTGTGGAGTGCCCAC | 47: CACAGATCCGGCTCTCTGTAGATTCAGGC |

TABLE 5
Sequence of each fusion protein

| | Number of | | |
|---|---|---|---|
| | amino acid | | |
| | residues in the | DNA | Amino acid |
| Fusion protein | mucin domain | sequence | sequence |
| hTim4-Fc | 181 | SEQ ID NO: 48 | SEQ ID NO: 49 |
| hTim4-240-Fc | 111 | SEQ ID NO: 50 | SEQ ID NO: 51 |
| hTim4-Δ131- | 128 | SEQ ID NO: 52 | SEQ ID NO: 53 |
| 187-Fc | | | |
| hTim4-Δ131- | 55 | SEQ ID NO: 54 | SEQ ID NO: 55 |
| 240-Fc | | | |
| hTim4-Δ131- | 58 | SEQ ID NO: 56 | SEQ ID NO: 57 |
| 187Δ241-310-Fc | | | |
| hTim4-187-Fc | 58 | SEQ ID NO: 58 | SEQ ID NO: 59 |
| hTim4-184-Fc | 55 | SEQ ID NO: 60 | SEQ ID NO: 61 |
| hTim4-181-Fc | 52 | SEQ ID NO: 62 | SEQ ID NO: 63 |
| hTim4-178-Fc | 49 | SEQ ID NO: 64 | SEQ ID NO: 65 |
| hTim4-175-Fc | 46 | SEQ ID NO: 66 | SEQ ID NO: 67 |
| hTim4-162-Fc | 33 | SEQ ID NO: 68 | SEQ ID NO: 69 |
| hTim4-149-Fc | 20 | SEQ ID NO: 70 | SEQ ID NO: 71 |
| hTim4-135-Fc | 6 | SEQ ID NO: 72 | SEQ ID NO: 73 |
| hTim4-130-Fc | 0 | SEQ ID NO: 74 | SEQ ID NO: 75 |

TABLE 6
Number of amino acid residues and molecular weight of
each fusion protein

| | Number of | Total | |
|---|---|---|---|
| | amino acid | number of | Molecular |
| | residues in the | amino acid | weight |
| Fusion protein | mucin domain | residues | (kDa) |
| hTim4-Fc | 181 | 534 | 276 |
| hTim4-240-Fc | 111 | 464 | 194 |
| hTim4-Δ131-187-Fc | 128 | 481 | 220 |
| hTim4-Δ131-240-Fc | 55 | 408 | 141 |
| hTim4-Δ131-187Δ241-310-Fc | 58 | 411 | 176 |
| hTim4-187-Fc | 58 | 411 | 175 |
| hTim4-184-Fc | 55 | 408 | 173 |
| hTim4-181-Fc | 52 | 405 | 164 |
| hTim4-178-Fc | 49 | 402 | 157 |
| hTim4-175-Fc | 46 | 399 | 157 |
| hTim4-162-Fc | 33 | 386 | 132 |
| hTim4-149-Fc | 20 | 373 | 125 |
| hTim4-135-Fc | 6 | 359 | 82 |
| hTim4-130-Fc | 0 | 354 | 79 |

### Reference Example 3

### Evaluation of Binding Capacity of Wild-Type or Modified hTim4-Fc to PS (1)

Dot blotting was carried out by the following procedure to evaluate the binding capacity of each fusion protein (hTim4-Fc) prepared as described above to PS.

On a PVDF membrane (GE Healthcare) with a size of 10 mm×10 mm, 0.15 μg of PS was spotted, and the resulting membrane was incubated in 500 μL of a blocking buffer (5% skim milk—PBS-T) in a 24-well plate at room temperature for 1 hour, to perform blocking. To the blocking buffer in the well, each fusion protein was added at a final concentration of 0.5 μg/mL, and incubation was carried out at 4° C. overnight. A control was provided by use of only the blocking buffer. Thereafter, the PVDF membrane was washed 5 times with 2 mL of PBS-T, and incubated at room temperature for 2 hours in an HRP-conjugated anti-human IgG-Fc antibody (Bethyl) solution which was 10000-fold diluted with 500 μL of the blocking buffer. Subsequently, the membrane was washed 5 times with 2 mL of PBS-T, followed by chemiluminescence using ECL prime (GE healthcare) and detection using LAS 3000 (Fujifilm).

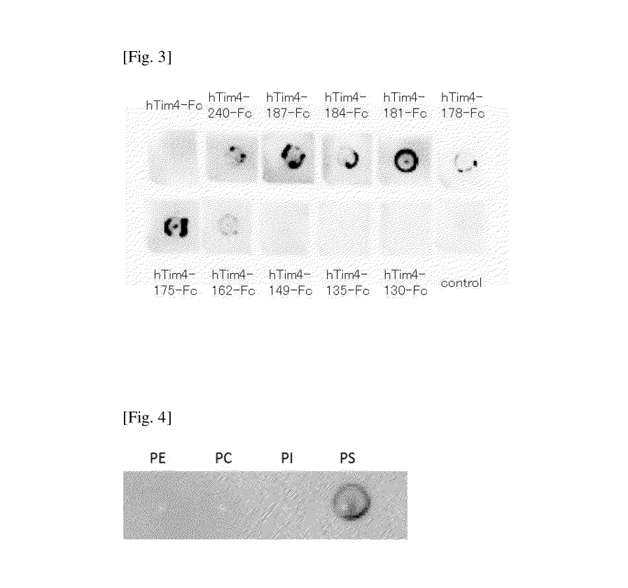

As can be seen from the results shown in FIG. 3, binding to PS was confirmed for hTim4-240-Fc, hTim4-187-Fc, hTim4-184-Fc, hTim4-181-Fc, hTim4-178-Fc, hTim4-175-Fc, and hTim4-162-Fc.

### Reference Example 4

### Evaluation of Binding Capacity of Wild-Type or Modified hTim4-Fc to PS (2)

ELISA was carried out by the following procedure to evaluate the binding capacity of each fusion protein (hTim4-Fc) prepared as described above to PS.

Into a 96-well plate, 100 μL of PS (0.5 mg/mL) or a solvent (methanol) was placed, and dried. Using 200 μL of 0.5% casein-PBS, blocking was performed at room temperature for 2 hours or at 4° C. overnight. A 10-fold dilution series of the sample was prepared, followed by carrying out the primary reaction. An HRP-conjugated anti-human IgG-Fc antibody was used as a secondary antibody, and the absorbance due to coloring with TMB was measured using a microplate reader. Based on the measurement results, the maximum binding amount (Bmax) and the 50% effective concentration (EC₅₀) were calculated using calculation software, Prism ver. 5.04. The results are shown in Table 7.

TABLE 7
Binding capacity of each fusion protein to PS as
measured by ELISA

| | Bmax ± SE (Abs | |
|---|---|---|
| Fusion protein | 450 nm) | EC₅₀ ± SE (nM) |
| hTim4-Fc | 0.00 ± 0.00 | 0.2 ± 0.9 |
| hTim4-240-Fc | 0.01 ± 0.01 | 7.1 ± 14.9 |
| hTim4-Δ131-187-Fc | — | — |
| hTim4-Δ131-240-Fc | 0.07 ± 0.02 | 147.9 ± 85.8 |
| hTim4-Δ131-187Δ241-310-Fc | 0.02 ± 0.03 | 131.6 ± 304.6 |
| hTim4-187-Fc | 1.02 ± 0.14 | 62.8 ± 20.2 |
| hTim4-184-Fc | 0.84 ± 0.17 | 100.1 ± 45.2 |
| hTim4-181-Fc | 0.74 ± 0.04 | 47.5 ± 6.2 |
| hTim4-178-Fc | 0.63 ± 0.22 | 67.0 ± 54.8 |
| hTim4-175-Fc | 0.77 ± 0.08 | 76.2 ± 19.5 |
| hTim4-162-Fc | 0.63 ± 0.44 | 193.9 ± 244.6 |
| hTim4-149-Fc | 0.03 ± 0.05 | 232.6 ± 550.8 |
| hTim4-135-Fc | 0.02 ± 0.01 | 26.7 ± 60.9 |
| hTim4-130-Fc | −0.01 ± 0.01 | 98.3 ± 233.0 |

Bmax: Maximum binding amount,
EC₅₀: 50% Effective concentration,
SE: Standard error,
—: Failed to calculate, n = 2

### Reference Example 5

### Evaluation of Binding Specificity of Modified hTim4-Fc to PS

Dot blotting was carried out by the following procedure to evaluate the binding capacity of the fusion protein of the present invention to various phospholipids.

On a PVDF membrane (GE Healthcare), 1 μL each of 100 pmol/μL 3-sn-phosphatidylethanolamine (PE), L-α-phosphatidylcholine (PC), L-α-phosphatidylinositol (PI), and 3-sn-phosphatidyl-L-serine (Sigma Aldrich) were spotted, and the resulting membrane was incubated in 2 mL of a blocking buffer (5% skim milk—PBS-T) at room temperature for 1 hour, to perform blocking. To the blocking buffer in each well, hTim4-187-Fc was added at a final concentration of 0.5 μg/mL, and incubation was carried out at room temperature for 2 hours. Thereafter, the PVDF membrane was washed 5 times with 5 mL of PBS-T, and incubated at room temperature for 2 hours in an HRP-conjugated anti-human IgG-Fc antibody (Bethyl) solution which was 10000-fold diluted with 2 mL of the blocking buffer. Subsequently, the membrane was washed 5 times with 5 mL of PBS-T, followed by chemiluminescence using ECL prime (GE healthcare) and detection using LAS 3000 (Fujifilm).

As can be seen from the results shown in FIG. 4, it was confirmed that hTim4-187-Fc binds to only PS.

### Reference Example 6

### Detection of Cells in which Apoptosis was Induced

Immunostaining was carried out by the following procedure to evaluate the binding capacity of hTim4-187-Fc or Annexin A5 to cells in which apoptosis was induced.

To 1×10⁶ cells/mL of Jurkat cells, Etoposid was added at a final concentration of 100 μM, and the cells were cultured under 5% CO₂ at 37° C. for 6 hours to induce apoptosis. The cells were collected and washed with cold PBS, and then suspended in 2% FBS-PBS at a density of 1×10⁷ cells/mL. Immunostaining with hTim4-187-Fc was carried out by adding hTim4-187-Fc, a CF488-conjugated anti-human IgG-Fc antibody (Biotium), and a propidium iodide (PI) solution (BioVision) to the above cell suspension at final concentrations of 0.5 μg/mL, 2.5 ng/mL, and 20 μL/mL, respectively, and then incubating the resulting mixture at room temperature for 5 minutes. Thereafter, the cells were washed with cold PBS and observed under a fluorescence microscope. Staining with Annexin A5 was carried out by using Annexin V-FITC Apoptosis Detection Kit (Bio Vision) for the above cell suspension, and the cells were similarly observed under a fluorescence microscope.

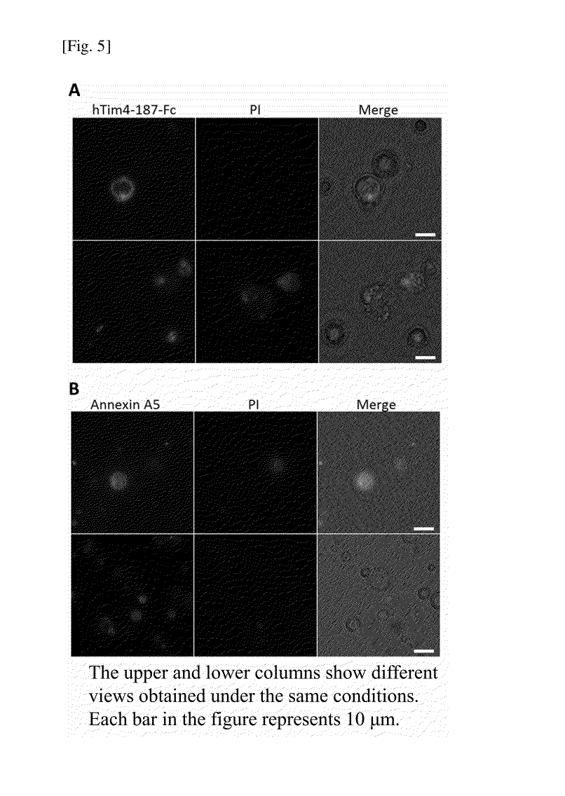

The results are shown in FIG. 5. It was confirmed that, similarly to the known probe Annexin A5, hTim4-187-Fc shows a binding capacity to cells in which apoptosis was induced, and does not bind to living cells. In addition, while the PI positivity indicates dead cells, it was also confirmed that, similarly to Annexin A5, hTim4-187-Fc detects both cells at a stage where the cells are dying due to apoptosis induction, and cells that have already died.

### INDUSTRIAL APPLICABILITY

According to the present invention, provided is a molecular imaging probe that enables highly sensitive detection of a dead cell(s), accumulates at a tumor site exhibiting cell death in vivo, and enables quantitative analysis. Thus, the present invention enables accurate detection and imaging of cell death in vivo, and use of the obtained result(s) for diagnosis of a disease or evaluation of a therapeutic effect of a drug, and hence, the present invention is industrially very useful.

While the present invention has been disclosed in detail with reference to preferred embodiments thereof, the present invention is not limited thereto. It will be apparent to one skilled in the art that modification(s) and/or alteration(s) may be made without departing from the gist and the scope of the present invention. Accordingly, the scope of the present invention should be defined by the later-described claims. All the cited references herein are incorporated as a part of this application by reference.

What is claimed is:

1. A protein as a probe for detecting an apoptotic cell(s) and/or a necrotic cell(s),
which is a fusion protein of a Tim4 protein and a protein or polypeptide to ensure dimerization, wherein the protein or polypeptide is fused to the C-terminus of the Tim4 protein,
wherein a part or the whole of the region consisting of the mucin domain, transmembrane domain, and cytoplasmic region in the Tim4 protein is replaced with a polypeptide consisting of the amino acid sequence of a) or b) below:
a) an amino acid sequence having a length of 30 to 120 amino acid residues consisting of a part of the amino acid sequence of the mucin domain of a wild-type Tim4 protein;
b) an amino acid sequence having an identity of not less than 80% to the amino acid sequence of a).

2. The protein according to claim 1, wherein the part or the whole of the region is replaced with a polypeptide consisting of the amino acid sequence of a′) or b′) below:
a′) an amino acid sequence from the amino acid residue at the N-terminus to the amino acid residue at any one of positions 30 to 120 of the amino acid sequence of the mucin domain of a wild-type Tim4 protein;
b′) an amino acid sequence having an identity of not less than 80% to the amino acid sequence of a′).

3. The protein according to claim 1, wherein the IgV domain of the Tim4 protein has the amino acid sequence of c) or d) below:
c) the amino acid sequence of the IgV domain of a wild-type Tim4 protein;
d) an amino acid sequence which is identical to the amino acid sequence of c) except that one or several amino acids are substituted, deleted, inserted, and/or added, and which has PS-binding capacity.

4. The protein according to claim 1, wherein the protein or polypeptide that forms a dimer is a human IgG Fc region protein.

5. The protein according to claim 1, wherein the wild-type Tim4 protein is a human Tim4 protein.

6. The protein according to claim 1, wherein the molecular weight of the fusion protein as measured by SDS-PAGE under non-reducing conditions is 100 kDa to 250 kDa.

7. A probe, comprising the protein according to claim 1, and being labeled.

8. A diagnostic imaging agent for a tumor, comprising the probe according to claim 7.

9. A diagnostic imaging kit, comprising the diagnostic imaging agent for a tumor according to claim 8.

10. A method for detecting an apoptotic cell(s) and/or a necrotic cell(s), comprising:
detecting an apoptotic cell(s) and/or a necrotic cell(s) by using the probe according to claim 7.

11. The protein according to claim 1, wherein the fusion protein is hTim4-Δ131-187-Fc.


SEQUENCE LISTING (75 sequences)

SEQ ID NO: 1 - DNA, 1306 bp, Homo sapiens
  CDS (58)..(1191)
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttaaaaaaaaaaaaaaa

SEQ ID NO: 2 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSAESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 3 - DNA, 2175 bp, Mus musculus
  CDS (14)..(1042)
agatcctatcaaaatgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcagatcaacagcagacagaccatcttgatcattgcctgctgtgtgggatttgtgctaatggtgttattgtttctggcgtttctccttcgagggaaagtcacaggagccaactgtttgcagagacacaagaggccagacaacactgaagatagtgacagcgtcctcaatgacatgtcacacgggagggatgatgaagacgggatcttcactctctgactcaccatctttatttaggattaaggatagggaatggcacttgaattgtcaaaataagtttggggacattgtaatttccgtttaaagtctcactctgtttactgatgctgtgggtcctgtctggttgtatcttcccacatgaaggtgctttagagacacattttccctgcctcgtgccttagtcctctttgttgttttgtggctaggtgacttttcacactgggcttgaacactgtcagtgatggtgaaatccttgccacagctttgggagtctcttgcagtctcccagcagtagagggagttagaaatatccagaggggaaaaaaaaatctctcttttcagacagtatctgctttattggtggtagctgaacttcatttatacagagctcctttaacctgtctgtcttcttcttggtatctaagctgccttttgtttttgtttttgtttttgtttttatgatattaacttcttttcacattcaagtttctttaaagttgactatagtgccttctgaactcttgcagagagtttggattttggaagctgccaggtacccatcacagcaggggtgccagtgacaaggatggtgtacaaatgaaacactgaagctatccaaataaattcctctaagtgtaattcattttactgcagcacaggaagaacaaatttgtcttacaactttaataattagtaccattatgaaccctaggagagaaataagagcaaatacctgttgaataaatgaatgtaagaaaatgtgtgtctgagcaagaatactctgtctggctactatgggaagctagctagatctgaaagacattctcagactatcctcatgttcaaggcattaaaggaataagcctccagcccctaaccttaggagaattctgcagtcaagtgaggagtttttaaaacaggaatctctaggttccagtcctctagctattcttttatgcttagtccaggtaatgagttgaacatccaagtattttttaaggacccaaagaaatgcaaccagagctattaccagaattttggagtggtcctcctagagttgccgcatgttgctgggaaaattggggtcttagagttcttagtctacttaataaaagaattttaaaaaatgg

SEQ ID NO: 4 - protein, 343 aa, Mus musculus
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEDSDSVLNDMSHGRDDEDGIFTL

SEQ ID NO: 5 - DNA, 540 bp, Homo sapiens
  CDS (1)..(540)
tcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaa

SEQ ID NO: 6 - protein, 180 aa, Homo sapiens
STTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSAESTSADTVLLTSKESKVWDLPSTSHVSMWKTSDSVSSPQPGASDTAVPEQNKTTKTGQMDGIPMSMKNEMPISQ

SEQ ID NO: 7 - DNA, 417 bp, Mus musculus
  CDS (1)..(417)
acaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcag

SEQ ID NO: 8 - protein, 139 aa, Mus musculus
TTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQ

SEQ ID NO: 9 - DNA, 798 bp, Artificial Sequence
  Description of Artificial Sequence Synthetic mTim4-Fc polynucleotide
atgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcag

SEQ ID NO: 10 - protein, 266 aa, Artificial Sequence
  Description of Artificial Sequence Synthetic mTim4-Fc polypeptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQ

SEQ ID NO: 11 - DNA, 28 bp, Artificial Sequence
  Description of Artificial Sequence Synthetic forward primer (mTim4-Fc)
atccttcgggccccccctcgaggtcgac

SEQ ID NO: 12 - DNA, 37 bp, Artificial Sequence
  Description of Artificial Sequence Synthetic reverse primer (mTim4-deltaM230-Fc)
ccgatatcggcttactagatatggtcatcatgcttct

SEQ ID NO: 13 - DNA, 37 bp, Artificial Sequence
  Description of Artificial Sequence Synthetic reverse primer (mTim4-deltaM184-Fc)
ccgatatcggcttactgaaggcagtggtggctagtgt

SEQ ID NO: 14 - DNA, 20 bp, Artificial Sequence
  Description of Artificial Sequence Synthetic forward primer (colony PCR)
ctgagtgggtggagactgaa

SEQ ID NO: 15 - DNA, 19 bp, Artificial Sequence
  Description of Artificial Sequence Synthetic reverse primer (colony PCR)
tctccctgagcatgagtgg

SEQ ID NO: 16 - DNA, 690 bp, Artificial Sequence
  Description of Artificial Sequence Synthetic mTim4-deltaM230-Fc polynucleotide
atgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatct

SEQ ID NO: 17 - protein, 230 aa, Artificial Sequence
  Description of Artificial Sequence Synthetic mTim4-deltaM230-Fc polypeptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTIS

SEQ ID NO: 18 - DNA, 552 bp, Artificial Sequence
  Description of Artificial Sequence Synthetic mTim4-deltaM184-Fc polynucleotide
atgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttc

SEQ ID NO: 19 - protein, 184 aa, Artificial Sequence
  Description of Artificial Sequence Synthetic mTim4-deltaM184-Fc polynucleotide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAF

SEQ ID NO: 20 - DNA, 24 bp, Artificial Sequence
  Description of Artificial Sequence Synthetic forward primer (hTim4-Fc plasmid)
accatgtacaggatgcaactcctg

SEQ ID NO: 21 - DNA, 24 bp, Artificial Sequence
  Description of Artificial Sequence Synthetic reverse primer (hTim4-Fc plasmid)
tccctgtctccgggtaaatgagtg

SEQ ID NO: 22 - DNA, 37 bp, Artificial Sequence
  Description of Artificial Sequence Synthetic forward primer (hTim4-Fc)
ttgatatccgagactgttgtgacggaggttttgggtc

SEQ ID NO: 23 - DNA, 37 bp, Artificial Sequence
  Description of Artificial Sequence Synthetic reverse primer (hTim4-Fc)
aaagatctttgggagatgggcatttcattcttcattg

SEQ ID NO: 24 - DNA, 37 bp, Artificial Sequence; identical to SEQ ID NO: 22
  Description of Artificial Sequence Synthetic forward primer (hTim4-240-Fc)

SEQ ID NO: 25 - DNA, 37 bp, Artificial Sequence
  Description of Artificial Sequence Synthetic reverse primer (hTim4-240-Fc)
acagatctagaagtagactcagcactactccaggaat

SEQ ID NO: 26 - DNA, 25 bp, Artificial Sequence
  Description of Artificial Sequence Synthetic forward primer (hTim4-delta131-187-Fc, hTim4-delta131-240-Fc)
gagagccccatcaacatcccacgtg

SEQ ID NO: 27 - DNA, 26 bp, Artificial Sequence
  Description of Artificial Sequence Synthetic reverse primer (hTim4-delta131-187-Fc, hTim4-delta131-240-Fc)
gttgatggggctctctgtagattcag

SEQ ID NO: 28 - DNA, 25 bp, Artificial Sequence
  Description of Artificial Sequence Synthetic forward primer (hTim4-delta131-187delta241-310-Fc)
gagagccattgccgtcttcacaaca

SEQ ID NO: 29 - DNA, 26 bp, Artificial Sequence
  Description of Artificial Sequence Synthetic reverse primer (hTim4-delta131-187delta241-310-Fc)
acggcaatggctctctgtagattcag

SEQ ID NO: 30 - DNA, 25 bp, Artificial Sequence
  Description of Artificial Sequence Synthetic forward primer (hTim4-187-Fc)
gtcttcggatctgtggagtgcccac

SEQ ID NO: 31 - DNA, 26 bp, Artificial Sequence
  Description of Artificial Sequence Synthetic reverse primer (hTim4-187-Fc)
cacagatccgaagacggcaatggttg

SEQ ID NO: 32 - DNA, 26 bp, Artificial Sequence
  Description of Artificial Sequence Synthetic forward primer (hTim4-184-Fc)
aaccattggatctgtggagtgcccac

SEQ ID NO: 33 - DNA, 29 bp, Artificial Sequence
  Description of Artificial Sequence Synthetic reverse primer (hTim4-184-Fc)
cacagatccaatggttgtcatctggagtg

SEQ ID NO: 34 - DNA, 27 bp, Artificial Sequence
  Description of Artificial Sequence Synthetic forward primer (hTim4-181-Fc)
tccagatgggatctgtggagtgcccac

SEQ ID NO: 35 - DNA, 29 bp, Artificial Sequence
  Description of Artificial Sequence Synthetic reverse primer (hTim4-181-Fc)
cacagatcccatctggagtggtgttccgg

SEQ ID NO: 36 - DNA, 26 bp, Artificial Sequence
  Description of Artificial Sequence Synthetic forward primer (hTim4-178-Fc)
aacaccaggatctgtggagtgcccac

SEQ ID NO: 37 - DNA, 29 bp, Artificial Sequence
  Description of Artificial Sequence Synthetic reverse primer (hTim4-178-Fc)
cacagatccaggtgttccggttgtgagat

SEQ ID NO: 38 - DNA, 26 bp, Artificial Sequence
  Description of Artificial Sequence Synthetic forward primer (hTim4-175-Fc)
cacaaccggatctgtggagtgcccac

SEQ ID NO: 39 - DNA, 29 bp, Artificial Sequence
  Description of Artificial Sequence Synthetic reverse primer (hTim4-175-Fc)
cacagatccggttgtgagatcgggtgtgg

SEQ ID NO: 40 - DNA, 26 bp, Artificial Sequence
  Description of Artificial Sequence Synthetic forward primer (hTim4-162-Fc)
agctgcaggatctgtggagtgcccac

SEQ ID NO: 41 - DNA, 28 bp, Artificial Sequence
  Description of Artificial Sequence Synthetic reverse primer (hTim4-162-Fc)
cacagatcctgcagctggggttgttgtc

SEQ ID NO: 42 - DNA, 25 bp, Artificial Sequence
  Description of Artificial Sequence Synthetic forward primer (hTim4-149-Fc)
acaagcggatctgtggagtgcccac

SEQ ID NO: 43 - DNA, 25 bp, Artificial Sequence
  Description of Artificial Sequence Synthetic reverse primer (hTim4-149-Fc)
cacagatccgcttgttgttgttgtt

SEQ ID NO: 44 - DNA, 25 bp, Artificial Sequence
  Description of Artificial Sequence Synthetic forward primer (hTim4-135-Fc)
acgcacggatctgtggagtgcccac

SEQ ID NO: 45 - DNA, 26 bp, Artificial Sequence
  Description of Artificial Sequence Synthetic reverse primer (hTim4-135-Fc)
cacagatccgtgcgtggttgttgagg

SEQ ID NO: 46 - DNA, 25 bp, Artificial Sequence
  Description of Artificial Sequence Synthetic forward primer (hTim4-130-Fc)
agagccggatctgtggagtgcccac

SEQ ID NO: 47 - DNA, 29 bp, Artificial Sequence
  Description of Artificial Sequence Synthetic reverse primer (hTim4-130-Fc)
cacagatccggctctctgtagattcaggc

SEQ ID NO: 48 - DNA, 1605 bp, Artificial Sequence
  Description of Artificial Sequence Synthetic hTim4-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacagggacagatggatggatacccatgtcaatgaagaatgaaatgcccatctcccaaagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 49 - protein, 534 aa, Artificial Sequence [sequence omitted]

SEQ ID NO: 50 - DNA, 1395 bp, Artificial Sequence
  Description of Artificial Sequence Synthetic hTim4-240-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctagatctgtggagtgcccaccttgcccagcaccacctgtggcaggaccttcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 51 - protein, 464 aa, Artificial Sequence
  Description of Artificial Sequence Synthetic hTim4-240-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSAESTSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 1446 bp, Artificial Sequence
  Description of Artificial Sequence Synthetic hTim4-delta131-187-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacagggacagatggatggatacccatgtcaatgaagaatgaaatgcccatctcccaaagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 53 - protein, 481 aa, Artificial Sequence
  Description of Artificial Sequence Synthetic hTim4-delta131-187-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAIAVFTTANTCLSLTPSTLPEEATGLLTPEPSKEGPILTAESETVLPSDSWSSAESTSADTVLLTSKESKVWDLPSTSHVSMWKTSDSVSSPQPGASDTAVPEQNKTTKTGTDGWIPMSMKNEMPISQRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1227 bp, Artificial Sequence
  Description of Artificial Sequence Synthetic hTim4-delta131-240-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagccccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaaagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 55 - protein, 408 aa, Artificial Sequence
  Description of Artificial Sequence Synthetic hTim4-delta131-240-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAPSTSHVSMWKTSDSVSSPQPGASDTAVPEQNKTTKTGQMDGIPMSMKNEMPISQRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1236 bp, Artificial Sequence
  Description of Artificial Sequence Synthetic hTim4-delta131-187delta241-310 polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgctgagtctacttctagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 57 - protein, 411 aa, Artificial Sequence
  Description of Artificial Sequence Synthetic hTim4-delta131-187delta241-310 polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAIAVFTTANTCLSLTPSTLPEEATGLLTPEPSKEGPILTAESETVLPSDSWSSAESTSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1236 bp, Artificial Sequence
  Description of Artificial Sequence Synthetic hTim4-187-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 59 - protein, 411 aa, Artificial Sequence
  Description of Artificial Sequence Synthetic hTim4-187-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1227 bp, Artificial Sequence
  Description of Artificial Sequence Synthetic hTim4-184-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 61 - protein, 408 aa, Artificial Sequence
  Description of Artificial Sequence Synthetic hTim4-184-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 1218 bp, Artificial Sequence
  Description of Artificial Sequence Synthetic hTim4-181-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 63 - protein, 405 aa, Artificial Sequence
  Description of Artificial Sequence Synthetic hTim4-181-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1209 bp, Artificial Sequence
  Description of Artificial Sequence Synthetic hTim4-178-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccaggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 65 - protein, 402 aa, Artificial Sequence
  Description of Artificial Sequence Synthetic hTim4-178-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1200 bp, Artificial Sequence
  Description of Artificial Sequence Synthetic hTim4-175-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 67 - protein, 399 aa, Artificial Sequence
  Description of Artificial Sequence Synthetic hTim4-175-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 1161 bp, Artificial Sequence
  Description of Artificial Sequence Synthetic hTim4-162-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcaggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 69 - protein, 386 aa, Artificial Sequence
  Description of Artificial Sequence Synthetic hTim4-162-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAAGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1122 bp, Artificial Sequence
  Description of Artificial Sequence Synthetic hTim4-149-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagcggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 71 - protein, 373 aa, Artificial Sequence
  Description of Artificial Sequence Synthetic hTim4-149-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1080 bp, Artificial Sequence
  Description of Artificial Sequence Synthetic hTim4-135-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 73 - protein, 359 aa, Artificial Sequence
  Description of Artificial Sequence Synthetic hTim4-135-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 1065 bp, Artificial Sequence
  Description of Artificial Sequence Synthetic hTim4-130-Fc polynucleotide
atgcaactcctgtcttgcattgcactaagtcttgcacttgtcacgaattcgatatccgagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagccggatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 75 - protein, 354 aa, Artificial Sequence
  Description of Artificial Sequence Synthetic hTim4-130-Fc polypeptide
MQLLSCIALSLALVTNSISETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAGSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK